(19) United States
(12) Patent Application Publication
(10) Pub. No.: US 2024/0006019 A1
(43) Pub. Date: Jan. 4, 2024

(54) METHODS FOR ASSESSING GENOMIC INSTABILITY

(71) Applicant: Life Technologies Corporation, Carlsbad, CA (US)

(72) Inventor: Mohit GUPTA, Dublin, CA (US)

(21) Appl. No.: 18/343,857

(22) Filed: Jun. 29, 2023

Related U.S. Application Data
(60) Provisional application No. 63/357,537, filed on Jun. 30, 2022.

(51) Int. Cl. G16B 20/10; G16B 20/20

(52) U.S. Cl. CPC G16B 20/10; G16B 20/20

(57) ABSTRACT

Methods for assessing genomic instability (GI) may include selectively amplifying nucleic acid sequences at targeted locations in a tumor sample genome by a targeted panel with a low sample input to generate a plurality of nucleic acid sequence reads; dividing the genome into segments having homogeneous copy numbers using log odds of heterozygous SNPs and CNV log ratios determined for the nucleic acid sequence reads; applying a threshold count to squared allelic log odds of each segment in autosomes of the genome to identify unbalanced copy number (UCN) segments; adding numbers of bases in the UCN segments to produce a sum of UCN bases; and dividing the sum of UCN bases by the total number of bases in all the segments identified in the autosomes of the sample genome to produce a ratio indicative of genomic instability. The ratio may be expressed as a percent to give a GI score.

### CROSS-REFERENCE TO RELATED APPLICATIONS

This application claims the benefit under 35 U.S.C. § 119(e) of U.S. Provisional Application No. 63/357,537, filed Jun. 30, 2022. The entire content of the aforementioned application is incorporated by reference herein.

### FIELD

The present disclosure relates to methods, systems, and computer-readable media for assessing genomic instability, and, more specifically, to methods, systems, and computer-readable media for assessing genomic instability in a tumor sample genome using nucleic acid sequencing data from targeted sequencing panels and next-generation sequencing (NGS) technology.

### BRIEF DESCRIPTION OF THE DRAWINGS

FIG. 1 is a block diagram of an example process for analyzing a sample genome to assess genomic instability.

FIG. 2A shows an example plot of CNV log ratios for a tumor sample genome.

FIG. 2B shows an example plot of log odds for the tumor sample genome.

FIG. 2C shows an example plot of copy numbers for each of the identified genomic segments in FIGS. 2A and 2B.

FIG. 3 shows an example of box plots for the genomic instability scores for BRCA1/2 mutations versus wild type BRCA1/2 where the size constraining step was not applied.

FIG. 4 shows an example of box plots for the genomic instability scores for BRCA1/2 mutations versus wild type BRCA1/2 where the size constraining step was applied.

FIG. 5 shows an example of box plots for the genomic instability scores for BRCA1/2 mutations versus wild type BRCA1/2 where the size constraining step was applied and excluding segments with UCN changes that are shorter than 10 Mb.

FIG. 6 shows an example of box plots for the genomic instability scores for BRCA1/2 mutations versus wild type BRCA1/2 where segments with UCN changes that have fewer than five heterozygous SNPs were excluded.

FIG. 7 shows an example of box plots for the genomic instability scores for BRCA1/2 mutations versus wild type BRCA1/2 where the weighted sum of UCN bases was calculated and divided by the total number of bases in all the segments identified in the autosomes.

FIG. 8 is a schematic diagram of an exemplary system for reconstructing a nucleic acid sequence, in accordance with various embodiments.

FIG. 9 is an example of a block diagram of an analysis pipeline for signal data obtained from a nucleic acid sequencing instrument.

### DETAILED DESCRIPTION

In accordance with the teachings and principles embodied in this application, new methods, systems and non-transitory machine-readable storage medium are provided to assess genomic instability by analysis of nucleic acid sequence reads from a tumor sample genome.

In various embodiments, DNA (deoxyribonucleic acid) may be referred to as a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. Certain pairs of nucleotides specifically bind to one another in a complementary fashion (called complementary base pairing). That is, adenine (A) pairs with thymine (T) (in the case of RNA, however, adenine (A) pairs with uracil (U)), and cytosine (C) pairs with guanine (G). When a first nucleic acid strand binds to a second nucleic acid strand made up of nucleotides that are complementary to those in the first strand, the two strands bind to form a double strand. In various embodiments, “nucleic acid sequencing data,” “nucleic acid sequencing information,” “nucleic acid sequence,” “genomic sequence,” “genetic sequence,” or “fragment sequence,” “nucleic acid sequence read” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

A “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, for example 3-4, to several hundreds of monomeric units. Whenever a polynucleotide such as an oligonucleotide is represented by a sequence of letters, such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

The phrase “next generation sequencing” or NGS refers to sequencing technologies having increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization.

The phrase “genomic variants” or “genome variants” denote a single or a grouping of sequences (in DNA or RNA) that have undergone changes as referenced against a particular species or sub-populations within a particular species due to mutations, recombination/crossover or genetic drift. Examples of types of genomic variants include, but are not limited to: single nucleotide polymorphisms (SNPs), copy number variations (CNVs), insertions/deletions (Indels), inversions, etc.

In various embodiments, genomic variants can be detected using a nucleic acid sequencing system and/or analysis of sequencing data. The sequencing workflow can begin with the test sample being sheared or digested into hundreds, thousands or millions of smaller fragments which are sequenced on a nucleic acid sequencer to provide hundreds, thousands or millions of sequence reads, such as nucleic acid sequence reads. Each read can then be mapped to a reference or target genome, and in the case of mate-pair fragments, the reads can be paired thereby allowing interrogation of repetitive regions of the genome. The results of mapping and pairing can be used as input for various standalone or integrated genome variant (for example, SNP, CNV, Indel, inversion, etc.) analysis tools.

The phrase “sample genome” can denote a whole or partial genome of an organism.

The term “allele” as used herein refers to a genetic variation associated with a gene or a segment of DNA, i.e., one of two or more alternate forms of a DNA sequence occupying the same locus.

The term “locus” as used herein refers to a specific position on a chromosome or a nucleic acid molecule. Alleles of a locus are located at identical sites on homologous chromosomes.

As used herein, a “targeted panel” refers to a set of target-specific primers that are designed for selective amplification of target gene sequences in a sample. In some embodiments, following selective amplification of at least one target sequence, the workflow further includes nucleic acid sequencing of the amplified target sequence.

As used herein, “target sequence” or “target gene sequence” and its derivatives, refers to any single or double-stranded nucleic acid sequence that can be amplified or synthesized according to the disclosure, including any nucleic acid sequence suspected or expected to be present in a sample. In some embodiments, the target sequence is present in double-stranded form and includes at least a portion of the particular nucleotide sequence to be amplified or synthesized, or its complement, prior to the addition of target-specific primers or appended adapters. Target sequences can include the nucleic acids to which primers useful in the amplification or synthesis reaction can hybridize prior to extension by a polymerase. In some embodiments, the term refers to a nucleic acid sequence whose sequence identity, ordering or location of nucleotides is determined by one or more of the methods of the disclosure.

As used herein, “target-specific primer” and its derivatives, refers to a single stranded or double-stranded polynucleotide, typically an oligonucleotide, that includes at least one sequence that is at least 50% complementary, typically at least 75% complementary or at least 85% complementary, more typically at least 90% complementary, more typically at least 95% complementary, more typically at least 98% or at least 99% complementary, or identical, to at least a portion of a nucleic acid molecule that includes a target sequence. In such instances, the target-specific primer and target sequence are described as “corresponding” to each other. In some embodiments, the target-specific primer is capable of hybridizing to at least a portion of its corresponding target sequence (or to a complement of the target sequence); such hybridization can optionally be performed under standard hybridization conditions or under stringent hybridization conditions. In some embodiments, the target-specific primer is not capable of hybridizing to the target sequence, or to its complement, but is capable of hybridizing to a portion of a nucleic acid strand including the target sequence, or to its complement. In some embodiments, a forward target-specific primer and a reverse target-specific primer define a target-specific primer pair that can be used to amplify the target sequence via template-dependent primer extension. Typically, each primer of a target-specific primer pair includes at least one sequence that is substantially complementary to at least a portion of a nucleic acid molecule including a corresponding target sequence but that is less than 50% complementary to at least one other target sequence in the sample. In some embodiments, amplification can be performed using multiple target-specific primer pairs in a single amplification reaction, wherein each primer pair includes a forward target-specific primer and a reverse target-specific primer, each including at least one sequence that substantially complementary or substantially identical to a corresponding target sequence in the sample, and each primer pair having a different corresponding target sequence.

Alterations in genes in the Homologous Recombination Repair (HRR) pathway may interfere with the ability to repair DNA double-strand breaks (DSBs), leading to Homologous Repair Deficiency (HRD). HRD is associated with sensitivity towards poly(ADP-ribose) polymerase inhibitors and cisplatin and its determination is used as a biomarker for therapy decision making Genomic instability is an emerging biomarker for HRD.

HRD is the inability of the cells to repair double stranded DNA breaks. It arises due to mutations in the genes in HRR pathway, especially the BRCA1 and BRCA2 genes. The consequence of HRD is accumulation of errors in the genome during cell division and DNA replication leading to a genomic scar. The genomic scar can be characterized by comprehensive profiling of the structural alterations, such as copy number changes, in the tumor genome. Therefore, there is a need for a comprehensive metric for assessing genomic instability.

A targeted panel with low sample input requirements may be used to assess genomic instability in a tumor sample. A targeted panel may provide a viable alternative to whole genome sequencing that may have higher input sample requirements. In some embodiments, the targeted panel may comprise the Oncomine Comprehensive Assay Plus, or OCA Plus panel, (Thermo Fisher Scientific). The OCA Plus panel interrogates 502 cancer-related genes. The OCA Plus panel has 1889 amplicons designed specifically to include heterozygous SNPs that have high minor allele frequencies and are spread evenly across the genome. In addition, the heterozygous SNPs present in the targeted medical content of the panel are also used. The heterozygous SNPs allows comprehensive profiling of the tumor sample for structural alterations, copy number (CN) changes and assessment of genomic instability. The OCA Plus panel may use a recommended amount of 20 ng, and as little as 10 ng, of nucleic acid isolated from formaldehyde fixed paraffin embedded (FFPE) tumor samples including fine needle biopsies. In some embodiments, the panel may comprise a custom panel or other targeted panel of cancer driver or other genes associated with cancer.

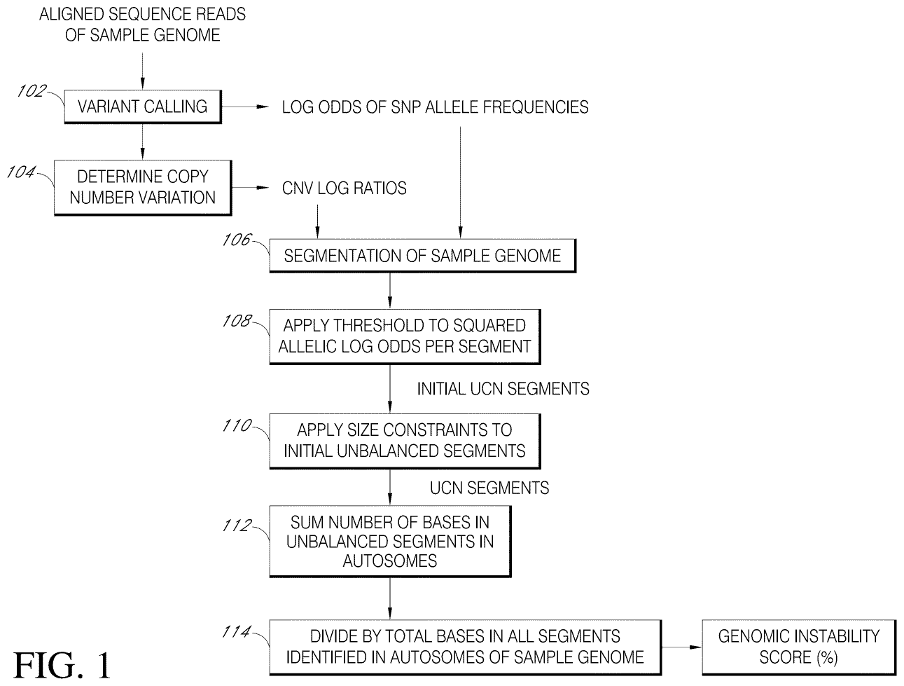

FIG. 1 is a block diagram of an example process for analyzing a sample genome to assess genomic instability. Selectively amplifying nucleic acid sequences at targeted locations in the tumor sample genome by a targeted panel with a low sample input from the tumor sample generates a plurality of nucleic acid sequence reads. The sequence reads are mapped to a reference genome to produce the aligned sequence reads. In the variant calling step 102, a processor receives aligned sequence reads resulting from targeted sequencing of a tumor sample. The aligned sequence reads can be retrieved from a file using a BAM file format, for example. The aligned sequence reads may correspond to a plurality of targeted locations in the tumor sample genome. The variant calling step 102 may be configured by one or more variant caller parameters. The variant calling step 102 may provide an observed population of variants, such as SNPs (single nucleotide polymorphism), detected in the aligned sequence reads. In addition, the variant calling step 102 may determine the log odds for variant allele frequency of observed population of SNPs. The log odds is calculated as the natural logarithm of the ratio of the number of sequence reads with the variant allele to the number of sequence reads with the reference allele. In some embodiments, the variant detection methods for use with the present teachings may include one or more features described in U.S. Pat. Appl. Publ. No. 2013/0345066, published Dec. 26, 2013, U.S. Pat. Appl. Publ. No. 2014/0296080, published Oct. 2, 2014, and U.S. Pat. Appl. Publ. No. 2014/0052381, published Feb. 20, 2014, each of which incorporated by reference herein in its entirety. Other variant detection methods may be used. In various embodiments, a variant caller can be configured to communicate variants called for a sample genome as a *.vcf, *.gff, or *.hdf data file. The called variant information can be communicated using any file format as long as the called variant information can be parsed and/or extracted for analysis. The copy number variation (CNV) step 104 may provide copy-number estimates and CNV log ratios of the aligned sequence reads. The CNV log ratios are calculated as the log 2 ratios of the copy-number estimates relative to the baseline copy number for each amplicon in the assay. In some embodiments, the CNV detection methods for use with the present teachings may include one or more features described in U.S. Pat. Appl. Publ. No. 2018/0268103, published Sep. 20, 2018, U.S. Pat. Appl. Publ. No. 2014/0256571, published Sep. 11, 2014, and U.S. Pat. Appl. Publ. No. 2016/0103957, published Apr. 14, 2016, each of which incorporated by reference herein in its entirety. Other CNV detection methods may be used. The segmentation step 106 uses the log odds and the CNV log ratios to divide the genome sequence into segments having homogeneous copy numbers. For example, the OCA Plus panel provides 1889 amplicons with heterozygous SNPs designed specifically to cover the genome and segment it for CN changes using joint segmentation of CNV log ratios and allelic log odds. The segmentation algorithm is circular binary segmentation that aims for joint segmentation of log 2 ratios and log odds to detect change points using Hotelling T2 statistic. (See e.g., R. Shen et al., FACETS: allele-specific copy number and clonal heterogeneity analysis tool for high-throughput DNA sequencing, Nucleic Acids Research, 2016, Vol. 44, No. 16 e131 doi: 10.1093/nar/gkw520). Optionally, the segmentation step 106, may exclude segments that have fewer than a minimum number of heterozygous SNPs. For example, the minimum number of SNPs in the segment may be set to a number in the range of 5 to 15 SNPs.

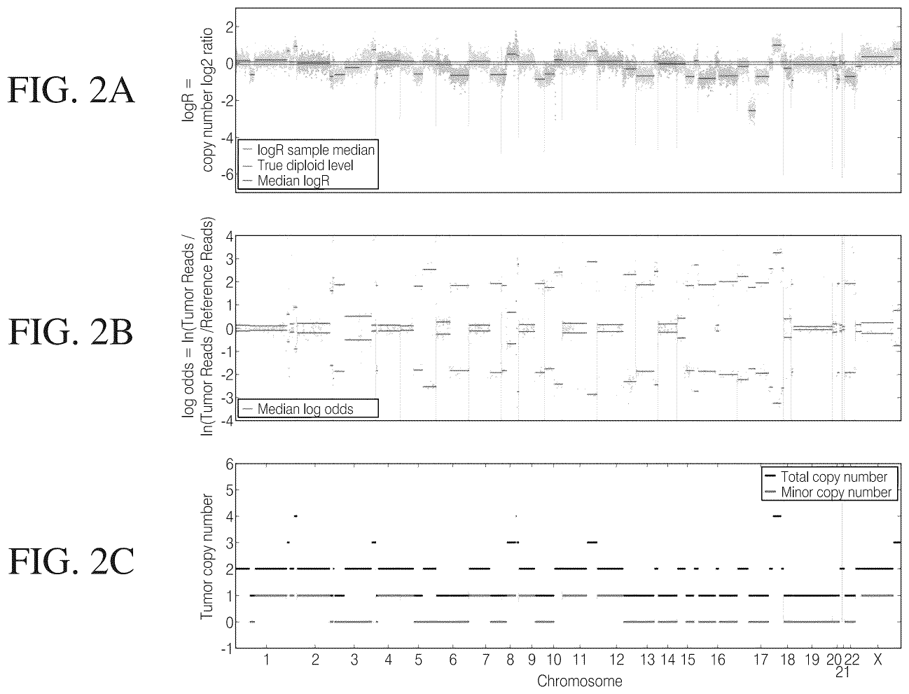

FIG. 2A shows an example plot of CNV log ratios for a tumor sample genome. Genome segmentation overlays (horizontal bars) show segments with similar log 2 ratios clustered together. FIG. 2B shows an example plot of log odds for the tumor sample genome. Genome segmentation overlays (horizontal bars) show segments with similar log odds clustered together. Since the variant allele could be major or minor for any SNP, the corresponding log odds could be positive or negative and are therefore displayed as segments that are mirror images around zero. FIG. 2C shows an example plot of copy numbers for each of the identified genomic segments in FIGS. 2A and 2B. The black horizontal bars indicate total copy numbers, and the red bars indicate minor copy numbers. Genomic segments where total CN≥1 and minor CN=0 are segments with loss of heterozygosity (LOH). There may exist genomic segments for which minor copy-number estimates cannot be determined.

Returning to FIG. 1, segments in autosomes with unbalanced copy numbers (UCN) are identified based on the allelic log odds corresponding to the individual segments defined by the segmentation step 106. The thresholding step 108 applies a threshold count to the squared allelic log odds of each segment to identify initial UCN segments in autosomes. The threshold count may be determined empirically. Example threshold count values are in a range from 0.05 to 0.26. For example, the threshold count value may be 0.05, 0.06, 0.07, 0.08, 0.09, 0.10, 0.11, 0.12, 0.13, 0.14, 0.15, 0.16, 0.17, 0.18, 0.19, 0.20, 0.21, 0.23, 0.24, 0.25, or 0.26. The size constraining step 110 may apply one or more size thresholds to the initial UCN segments. The size thresholds may include a maximum length threshold. The size constraining step 110 may exclude those initial UCN segments having lengths that span the maximum length threshold or more. For example, the size constraining step 110 may exclude initial UCN segments that fulfill one or more of the following conditions:
1) Span the maximum length threshold or more of the whole chromosome;
2) Span the maximum length threshold or more of the p-arm of the chromosome;
3) Span the maximum length threshold or more of the q-arm of the chromosome.

The values of the maximum length threshold may be different for the whole chromosome, the p-arm and the q-arm. The maximum length threshold value may be a percent of the whole chromosome length, a percent of the p-arm length or a percent of the q-arm length. For example, the maximum length threshold value may be set to 90%. For example, the maximum length threshold value may be at least 80%. The size thresholds may include a minimum length threshold. The size constraint step 110 may exclude those initial UCN segments having lengths that span the minimum length threshold or less. For example, the minimum length threshold may be 10 megabases (Mb). The minimum length threshold may be in a range of 5 to 15 Mb. The size constraining step 110 filters out any initial UCN segments not meeting the size constraint criteria to produce a set of UCN segments.

The summing step 112 may add the numbers of bases in the set of UCN segments in autosomes to produce a sum of UCN bases. The dividing step 114 may divide the sum of UCN bases by the total number of bases in all the segments identified in autosomes of the sample genome to produce a ratio. The ratio may be expressed as a percent to give a genomic instability (GI) score, or GI metric.

Alternatively, the summing step 112 may calculate a weighted sum of UCN bases. For example, the number of bases in each UCN segment in a given chromosome may be divided by the total number of bases in the chromosome containing the UCN segment to give a normalized number of bases per UCN segment. The normalized number of bases per UCN segment may be multiplied by a weight value to give a weighted number of bases per UCN segment. For example, the same weight value may be applied to the normalized number of bases per UCN segment for every UCN segment of a given chromosome. For example, the weight value may be a function of the number of UCN segments in the chromosome. For example, the weight value may be the number of UCN segments in the chromosome. The sum of the weighted number of bases per UCN segment for all the UCN segments for all the autosomes may be calculated to form the weighted sum of UCN bases. The dividing step 114 may divide the weighted sum of UCN bases by the total number of bases in all the segments identified in the autosomes of the sample genome to produce a ratio. The ratio may be expressed as a percent to give a genomic instability (GI) score, or GI metric.

To test the effectiveness of the methods described herein for assessing genomic instability, the genomic instability scores determined for ovarian tumor samples in an ovarian tumor FFPE cohort. The tumor samples having deleterious germline/somatic mutations BRCA1 and BRCA2 genes in the ovarian tumor FFPE cohort were compared to those tumor samples in the cohort having wild type (WT) BRCA1 and BRCA2 genes. The OCA Plus panel was used to generate targeted sequencing reads of the tumor samples in the cohort. The methods described with respect to FIG. 1 were applied to the aligned sequence reads corresponding to the tumor samples in the cohort. The number of tumor samples in the cohort, N=41. The results show that the tumor samples having deleterious germline/somatic mutations BRCA1 and BRCA2 genes have significantly higher GI score than the samples having WT BRCA1 and BRCA2 genes. These results indicate that GI can be used to characterize genomic scar. The results also suggest the targeted panel is sufficiently large to detect genomic instability.

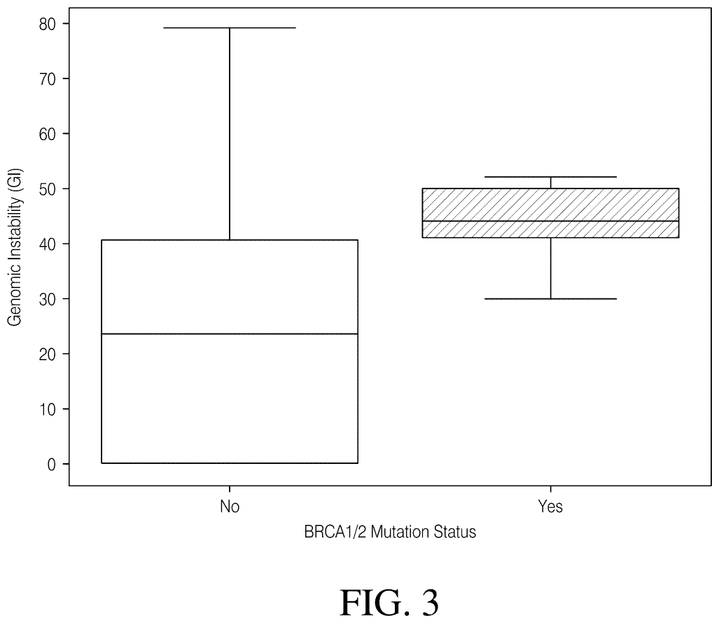

FIG. 3 shows an example of box plots for the genomic instability scores for BRCA1/2 mutations versus wild type BRCA1/2 where the size constraining step 110 was not applied. For this example, the size constraint step 110 was not applied, so initial UCN segments covering the whole chromosome, p-arm and q-arm were included for the summing step 112. These results show stratification of GI scores in BRCA1/2 mutated vs. wild type (WT). The p-value using student's double-sided t-test is 0.043714.

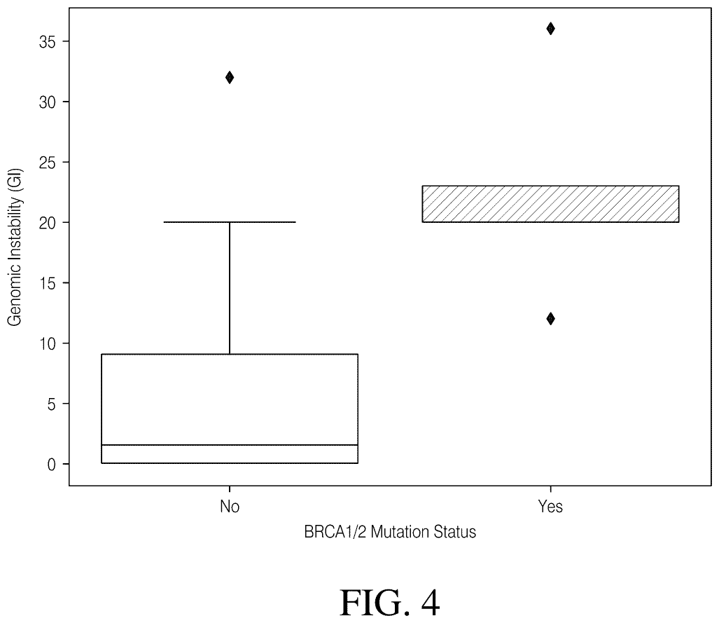

FIG. 4 shows an example of box plots for the genomic instability scores for BRCA1/2 mutations versus wild type BRCA1/2 where the size constraining step 110 was applied. For this example, the size constraint step 110 was applied, so initial UCN segments covering 90% or more of the whole chromosome, p-arm and q-arm were excluded for the summing step 112. These results show stratification of GI scores in BRCA1/2 mutated vs. wild type (WT). The p-value using student's double-sided t-test is 0.000086.

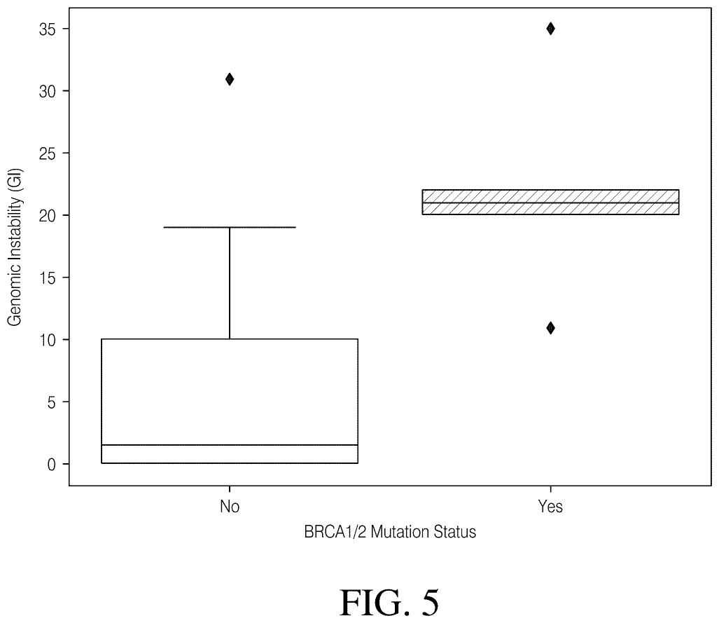

FIG. 5 shows an example of box plots for the genomic instability scores for BRCA1/2 mutations versus wild type BRCA1/2 where the size constraining step was applied and excluding segments with UCN changes that are shorter than 10 Mb. For this example, the size constraint step 110 was applied, so that initial UCN segments covering 90% or more of the whole chromosome, p-arm and q-arm were excluded and segments with UCN changes that are shorter than 10 Mb were excluded prior to the summing step 112. These results show stratification of GI scores in BRCA1/2 mutated vs. wild type (WT). The p-value using student's double-sided t-test is 0.000112.

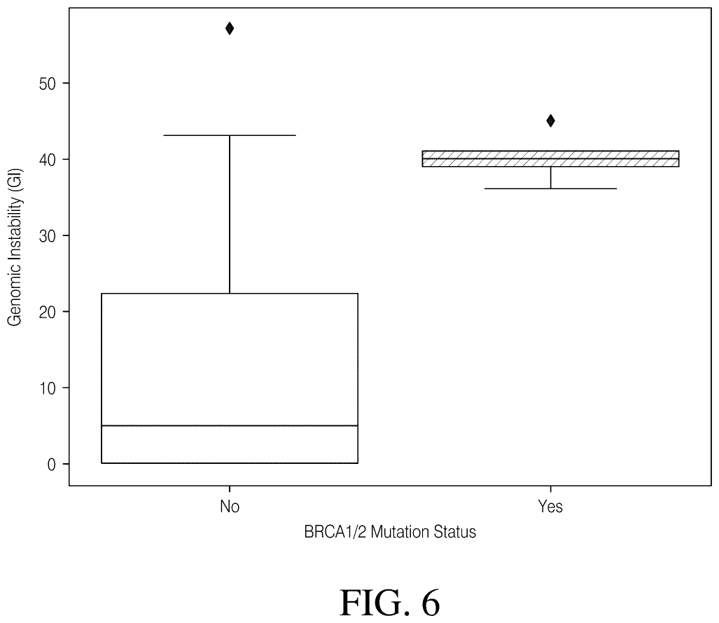

FIG. 6 shows an example of box plots for the genomic instability scores for BRCA1/2 mutations versus wild type BRCA1/2 where segments with UCN changes that have fewer than five heterozygous SNPs were excluded. For this example, the segmentation step 106 excluded segments that have fewer than five heterozygous SNPs. The size constraint step 110 was applied, so that initial UCN segments covering 90% or more of the whole chromosome, p-arm and q-arm were excluded and segments with UCN changes that are shorter than 10 Mb were excluded prior to the summing step 112. These results show stratification of GI scores in BRCA1/2 mutated vs. wild type (WT). The p-value using student's double-sided t-test is 0.000271.

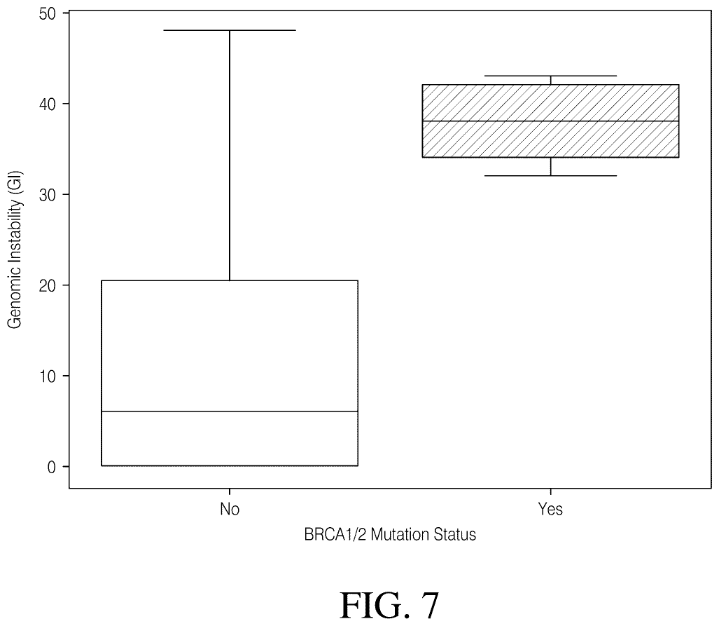

FIG. 7 shows an example of box plots for the genomic instability scores for BRCA1/2 mutations versus wild type BRCA1/2 where the weighted sum of UCN bases was calculated and divided by the total number of bases in all the segments identified in the autosomes. For this example, the size constraint step 110 was applied, so that initial UCN segments covering 90% or more of the whole chromosome, p-arm and q-arm were excluded and segments with UCN changes that are shorter than 10 Mb were excluded prior to the summing step 112. These results show stratification of GI scores in BRCA1/2 mutated vs. wild type (WT). The p-value using student's double-sided t-test is 0.000339.

The targeted panel and method for assessing genomic instability described herein provide improvements to the technology over whole genome sequencing (WGS). Sequence assembly methods must be able to assemble and/or map a large number of reads efficiently, such as by minimizing use of computational resources. For example, the sequencing of a human size genome can result in tens or hundreds of millions of reads that need to be assembled before they can be further analyzed. Computer processing of the nucleic acid sequence reads from targeted sequencing reduces computational requirements and memory requirements versus processing for WGS data. For WGS, 3 Gb of the tumor genome would be covered. The data resulting from the nucleic acid sequence reads for WGS would require computations and storage in memory for the nucleic acid sequence reads and variant data. In comparison, the targeted panel that covers approximately 1 Mb of the tumor genome would require substantially fewer computations and substantially less memory for storage of the nucleic acid sequence reads and variant data.

The targeted panel and method for assessing genomic instability for a tumor only sample described herein provide improvements to the technology over matched tumor-normal sample processing. In some cases, a matched normal sample for the tumor sample may not be available. When the matched normal sample is available, detecting variants and CNVs, in the nucleic acid sequence reads from the normal sample require at least the same amount of processing as for the tumor sample, thereby at least doubling the computations and memory requirements.

Example 1 is a method for analyzing a tumor sample genome for genomic instability, including: selectively amplifying nucleic acid sequences at targeted locations in the tumor sample genome by a targeted panel with a low sample input from a tumor sample to generate a plurality of nucleic acid sequence reads; dividing the genome into segments having homogeneous copy numbers using log odds of heterozygous SNPs and CNV log ratios determined for the plurality of nucleic acid sequence reads, wherein the heterozygous SNPs are distributed across the genome; applying a threshold count to squared allelic log odds of each segment in autosomes of the genome to identify unbalanced copy number (UCN) segments; adding numbers of bases in the UCN segments to produce a sum of UCN bases; and dividing the sum of UCN bases by the total number of bases in all the segments identified in the autosomes of the genome to produce a ratio indicative of genomic instability.

Example 2 includes the subject matter of any of Examples 1, and further specifies that the ratio is expressed as a percent to give a genomic instability (GI) score.

Example 3 includes the subject matter of any of Examples 1, and further includes applying a maximum length threshold to each of the UCN segments prior to the step of adding.

Example 4 includes the subject matter of any of Examples 3, and further includes excluding UCN segments that span the maximum length threshold or more of a whole chromosome.

Example 5 includes the subject matter of any of Examples 3, and further includes excluding UCN segments that span the maximum length threshold or more of a p-arm of a chromosome.

Example 6 includes the subject matter of any of Examples 3, and further includes excluding UCN segments that span the maximum length threshold or more of a q-arm of a chromosome.

Example 7 includes the subject matter of any of Examples 3, and further specifies that the maximum length threshold value is a percent of a whole chromosome length, a percent of a p-arm length or a percent of a q-arm length.

Example 8 includes the subject matter of any of Examples 7, and further specifies that the maximum length threshold value is 90%.

Example 9 includes the subject matter of any of Examples 1, and further includes applying a minimum length threshold to the UCN segments and excluding the UCN segments that span the minimum length threshold or less prior to the step of adding.

Example 10 includes the subject matter of any of Examples 9, and further specifies that the minimum length threshold is 10 megabases (Mb)

Example 11 includes the subject matter of any of Examples 1, and further includes dividing the number of bases in the UCN segment by a total number of bases in a chromosome containing the UCN segment to produce a normalized number of UCN bases per UCN segment.

Example 12 includes the subject matter of any of Examples 11, and further includes multiplying the normalized number of UCN bases per UCN segment by a weight value to give a weighted number of bases per UCN segment, wherein the step of adding is applied to the weighted number of bases per UCN segment for all the UCN segments to produce the sum of UCN bases.

Example 13 includes the subject matter of any of Examples 12, and further specifies that the weight value is a total number of UCN segments in the chromosome.

Example 14 includes the subject matter of any of Examples 1, and further includes excluding the segments having fewer than a minimum number of heterozygous SNPs in the segment.

Example 15 includes the subject matter of any of Examples 1, and further specifies that the ratio indicative of genomic instability is determined based on analyzing the tumor sample only.

Example 16 is a system for analyzing a tumor sample genome for genomic instability, including a processor and a data store communicatively connected with the processor, the processor configured to execute instructions, which, when executed by the processor, cause the system to perform a method, including: receiving a plurality of nucleic acid sequence reads generated by selectively amplifying nucleic acid sequences at targeted locations in the tumor sample genome by a targeted panel with a low sample input from a tumor sample; dividing the genome into segments having homogeneous copy numbers using log odds of heterozygous SNPs and CNV log ratios determined for the plurality of nucleic acid sequence reads, wherein the heterozygous SNPs are distributed across the genome; applying a threshold count to squared allelic log odds of each segment in autosomes of the genome to identify unbalanced copy number (UCN) segments; adding numbers of bases in the UCN segments to produce a sum of UCN bases; and dividing the sum of UCN bases by the total number of bases in all the segments identified in the autosomes of the genome to produce a ratio indicative of genomic instability.

Example 17 includes the subject matter of any of Examples 16, and further specifies that the ratio is expressed as a percent to give a genomic instability (GI) score.

Example 18 includes the subject matter of any of Examples 16, and further includes applying a maximum length threshold to each of the UCN segments prior to the step of adding.

Example 19 includes the subject matter of any of Examples 18, and further includes excluding UCN segments that span the maximum length threshold or more of a whole chromosome.

Example 20 includes the subject matter of any of Examples 18, and further includes excluding UCN segments that span the maximum length threshold or more of a p-arm of a chromosome.

Example 21 includes the subject matter of any of Examples 18, and further includes excluding UCN segments that span the maximum length threshold or more of a q-arm of a chromosome.

Example 22 includes the subject matter of any of Examples 18, and further specifies that the maximum length threshold value is a percent of a whole chromosome length, a percent of a p-arm length or a percent of a q-arm length.

Example 23 includes the subject matter of any of Examples 22, and further specifies that the maximum length threshold value is 90%.

Example 24 includes the subject matter of any of Examples 16, and further includes applying a minimum length threshold to the UCN segments and excluding the UCN segments that span the minimum length threshold or less prior to the step of adding.

Example 25 includes the subject matter of any of Examples 24, and further specifies that the minimum length threshold is 10 megabases (Mb)

Example 26 includes the subject matter of any of Examples 16, and further includes dividing the number of bases in the UCN segment by a total number of bases in a chromosome containing the UCN segment to produce a normalized number of UCN bases per UCN segment.

Example 27 includes the subject matter of any of Examples 26, and further includes multiplying the normalized number of UCN bases per UCN segment by a weight value to give a weighted number of bases per UCN segment, wherein the step of adding is applied to the weighted number of bases per UCN segment for all the UCN segments to produce the sum of UCN bases.

Example 28 includes the subject matter of any of Examples 27, and further specifies that the weight value is a total number of UCN segments in the chromosome.

Example 29 includes the subject matter of any of Examples 16, and further includes excluding the segments having fewer than a minimum number of heterozygous SNPs in the segment.

Example 30 includes the subject matter of any of Examples 16, and further specifies that the ratio indicative of genomic instability is determined based on analyzing the tumor sample only.

Example 31 is non-transitory computer-readable medium storing instructions that, when executed by a computer, cause the computer to perform a method for analyzing a tumor sample genome for genomic instability, the method including: receiving a plurality of nucleic acid sequence reads generated by selectively amplifying nucleic acid sequences at targeted locations in the tumor sample genome by a targeted panel with a low sample input from a tumor sample; dividing the genome into segments having homogeneous copy numbers using log odds of heterozygous SNPs and CNV log ratios determined for the plurality of nucleic acid sequence reads, wherein the heterozygous SNPs are distributed across the genome; applying a threshold count to squared allelic log odds of each segment in autosomes of the genome to identify unbalanced copy number (UCN) segments; adding numbers of bases in the UCN segments to produce a sum of UCN bases; and dividing the sum of UCN bases by the total number of bases in all the segments identified in the autosomes of the genome to produce a ratio indicative of genomic instability.

Example 32 includes the subject matter of any of Examples 31, and further specifies that the ratio is expressed as a percent to give a genomic instability (GI) score.

Example 33 includes the subject matter of any of Examples 31, and further includes applying a maximum length threshold to each of the UCN segments prior to the step of adding.

Example 34 includes the subject matter of any of Examples 33, and further includes excluding UCN segments that span the maximum length threshold or more of a whole chromosome.

Example 35 includes the subject matter of any of Examples 33, and further includes excluding UCN segments that span the maximum length threshold or more of a p-arm of a chromosome.

Example 36 includes the subject matter of any of Examples 33, and further includes excluding UCN segments that span the maximum length threshold or more of a q-arm of a chromosome.

Example 37 includes the subject matter of any of Examples 33, and further specifies that the maximum length threshold value is a percent of a whole chromosome length, a percent of a p-arm length or a percent of a q-arm length.

Example 38 includes the subject matter of any of Examples 37, and further specifies that the maximum length threshold value is 90%.

Example 39 includes the subject matter of any of Examples 31, and further includes applying a minimum length threshold to the UCN segments and excluding the UCN segments that span the minimum length threshold or less prior to the step of adding.

Example 40 includes the subject matter of any of Examples 39, and further specifies that the minimum length threshold is 10 megabases (Mb)

Example 41 includes the subject matter of any of Examples 31, and further includes dividing the number of bases in the UCN segment by a total number of bases in a chromosome containing the UCN segment to produce a normalized number of UCN bases per UCN segment.

Example 42 includes the subject matter of any of Examples 41, and further includes multiplying the normalized number of UCN bases per UCN segment by a weight value to give a weighted number of bases per UCN segment, wherein the step of adding is applied to the weighted number of bases per UCN segment for all the UCN segments to produce the sum of UCN bases.

Example 43 includes the subject matter of any of Examples 42, and further specifies that the weight value is a total number of UCN segments in the chromosome.

Example 44 includes the subject matter of any of Examples 31, and further includes excluding the segments having fewer than a minimum number of heterozygous SNPs in the segment.

Example 45 includes the subject matter of any of Examples 31, and further specifies that the ratio indicative of genomic instability is determined based on analyzing the tumor sample only.

In various embodiments, nucleic acid sequence data can be generated using various techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, fluorescent-based detection systems, single molecule methods, etc.

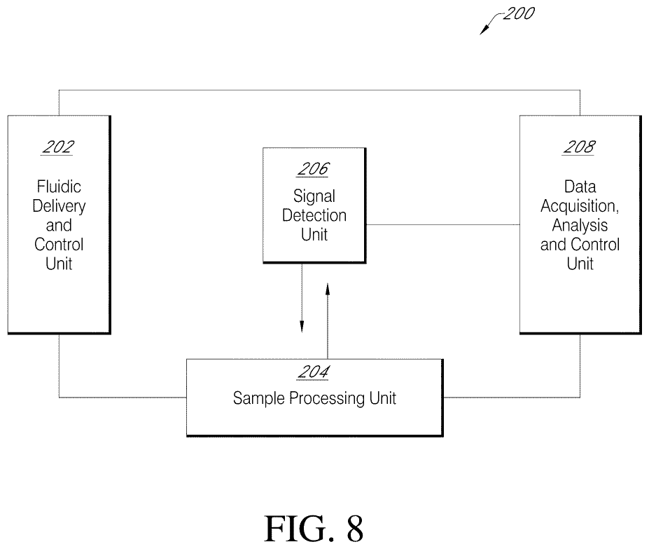

Various embodiments of nucleic acid sequencing platforms, such as a nucleic acid sequencer, can include components as displayed in the block diagram of FIG. 8. According to various embodiments, sequencing instrument 200 can include a fluidic delivery and control unit 202, a sample processing unit 204, a signal detection unit 206, and a data acquisition, analysis and control unit 208. Various embodiments of instrumentation, reagents, libraries and methods used for next generation sequencing are described in U.S. Patent Application Publication No. 2009/0127589 and No. 2009/0026082. Various embodiments of instrument 200 can provide for automated sequencing that can be used to gather sequence information from a plurality of sequences in parallel, such as substantially simultaneously.

In various embodiments, the fluidics delivery and control unit 202 can include reagent delivery system. The reagent delivery system can include a reagent reservoir for the storage of various reagents. The reagents can include RNA-based primers, forward/reverse DNA primers, oligonucleotide mixtures for ligation sequencing, nucleotide mixtures for sequencing-by-synthesis, optional ECC oligonucleotide mixtures, buffers, wash reagents, blocking reagent, stripping reagents, and the like. Additionally, the reagent delivery system can include a pipetting system or a continuous flow system which connects the sample processing unit with the reagent reservoir.

In various embodiments, the sample processing unit 204 can include a sample chamber, such as flow cell, a substrate, a micro-array, a multi-well tray, or the like. The sample processing unit 204 can include multiple lanes, multiple channels, multiple wells, or other means of processing multiple sample sets substantially simultaneously. Additionally, the sample processing unit can include multiple sample chambers to enable processing of multiple runs simultaneously. In particular embodiments, the system can perform signal detection on one sample chamber while substantially simultaneously processing another sample chamber. Additionally, the sample processing unit can include an automation system for moving or manipulating the sample chamber.

In various embodiments, the signal detection unit 206 can include an imaging or detection sensor. For example, the imaging or detection sensor can include a CCD, a CMOS, an ion sensor, such as an ion sensitive layer overlying a CMOS, a current detector, or the like. The signal detection unit 206 can include an excitation system to cause a probe, such as a fluorescent dye, to emit a signal. The expectation system can include an illumination source, such as arc lamp, a laser, a light emitting diode (LED), or the like. In particular embodiments, the signal detection unit 206 can include optics for the transmission of light from an illumination source to the sample or from the sample to the imaging or detection sensor. Alternatively, the signal detection unit 206 may not include an illumination source, such as for example, when a signal is produced spontaneously as a result of a sequencing reaction. For example, a signal can be produced by the interaction of a released moiety, such as a released ion interacting with an ion sensitive layer, or a pyrophosphate reacting with an enzyme or other catalyst to produce a chemiluminescent signal. In another example, changes in an electrical current can be detected as a nucleic acid passes through a nanopore without the need for an illumination source.

In various embodiments, data acquisition analysis and control unit 208 can monitor various system parameters. The system parameters can include temperature of various portions of instrument 200, such as sample processing unit or reagent reservoirs, volumes of various reagents, the status of various system subcomponents, such as a manipulator, a stepper motor, a pump, or the like, or any combination thereof.

It will be appreciated by one skilled in the art that various embodiments of instrument 200 can be used to practice variety of sequencing methods including ligation-based methods, sequencing by synthesis, single molecule methods, nanopore sequencing, and other sequencing techniques.

In various embodiments, the sequencing instrument 200 can determine the sequence of a nucleic acid, such as a polynucleotide or an oligonucleotide. The nucleic acid can include DNA or RNA, and can be single stranded, such as ssDNA and RNA, or double stranded, such as dsDNA or a RNA/cDNA pair. In various embodiments, the nucleic acid can include or be derived from a fragment library, a mate pair library, a ChIP fragment, or the like. In particular embodiments, the sequencing instrument 200 can obtain the sequence information from a single nucleic acid molecule or from a group of substantially identical nucleic acid molecules.

In various embodiments, sequencing instrument 200 can output nucleic acid sequencing read data in a variety of different output data file types/formats, including, but not limited to: *.fasta, *.csfasta, *seq.txt, *qseq.txt, *.fastq, *.sff, *prb.txt, *.sms, *srs and/or *.qv.

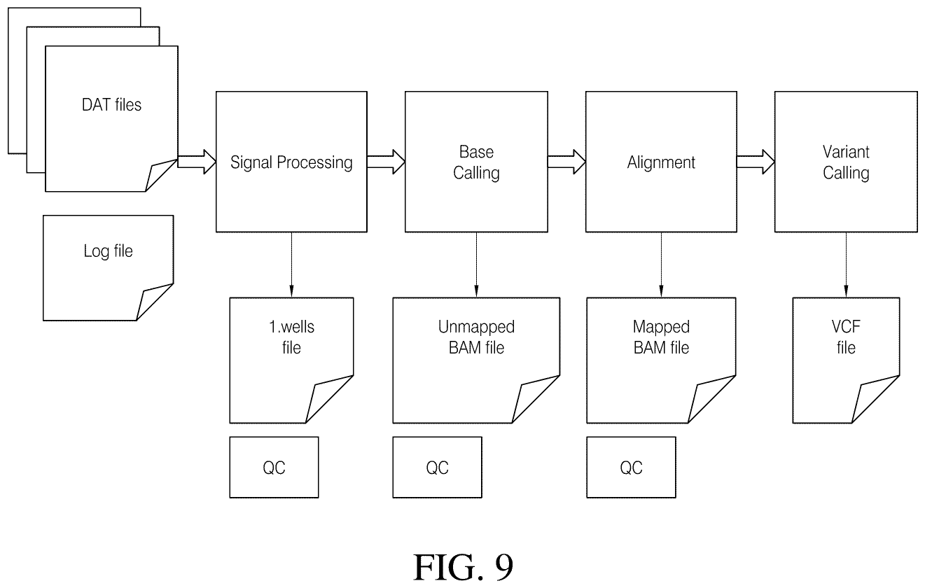

FIG. 9 is a block diagram of an analysis pipeline for signal data obtained from a nucleic acid sequencing instrument. The sequencing instrument generates raw data files (DAT, or .dat, files) during a sequencing run for an assay. Signal processing may be applied to raw data to generate incorporation signal measurement data for files, such as the 1.wells files, which are transferred to the server FTP location along with the log information of the run. The signal processing step may derive background signals corresponding to wells. The background signals may be subtracted from the measured signals for the corresponding wells. The remaining signals may be fit by an incorporation signal model to estimate the incorporation at each nucleotide flow for each well. The output from the above signal processing is a signal measurement per well and per flow, that may be stored in a file, such as a 1.wells file.

In some embodiments, the base calling step may perform phase estimations, normalization, and runs a solver algorithm to identify best partial sequence fit and make base calls. The base sequences for the sequence reads are stored in unmapped BAM files. The base calling step may generate total number of reads, total number of bases, and average read length as quality control (QC) measures to indicate the base call quality. The base calls may be made by analyzing any suitable signal characteristics (e.g., signal amplitude or intensity). The signal processing and base calling for use with the present teachings may include one or more features described in U.S. Pat. Appl. Publ. No. 2013/0090860 published Apr. 11, 2013, U.S. Pat. Appl. Publ. No. 2014/0051584 published Feb. 20, 2014, and U.S. Pat. Appl. Publ. No. 2012/0109598 published May 3, 2012, each incorporated by reference herein in its entirety.

Once the base sequence for the sequence read is determined, the sequence reads may be provided to the alignment step, for example, in an unmapped BAM file. The alignment step maps the sequence reads to a reference genome to determine aligned sequence reads and associated mapping quality parameters. The alignment step may generate a percent of mappable reads as QC measure to indicate alignment quality. The alignment results may be stored in a mapped BAM file. Methods for aligning sequence reads for use with the present teachings may include one or more features described in U.S. Pat. Appl. Publ. No. 2012/0197623, published Aug. 2, 2012, incorporated by reference herein in its entirety.

The BAM file format structure is described in “Sequence Alignment/Map Format Specification,” Sep. 12, 2014 (github.com/samtools/hts-specs). As described herein, a “BAM file” refers to a file compatible with the BAM format. As described herein, an “unmapped” BAM file refers to a BAM file that does not contain aligned sequence read information and mapping quality parameters and a “mapped” BAM file refers to a BAM file that contains aligned sequence read information and mapping quality parameters.

The variant calling step may include detecting single-nucleotide polymorphisms (SNPs), insertions and deletions (InDels), multi-nucleotide polymorphisms (MNPs), and complex block substitution events. In various embodiments, a variant caller can be configured to communicate variants called for a sample genome as a *.vcf, *.gff, or *.hdf data file. The called variant information can be communicated using any file format as long as the called variant information can be parsed and/or extracted for analysis. The variant detection methods for use with the present teachings may include one or more features described in U.S. Pat. Appl. Publ. No. 2013/0345066, published Dec. 26, 2013, U.S. Pat. Appl. Publ. No. 2014/0296080, published Oct. 2, 2014, and U.S. Pat. Appl. Publ. No. 2014/0052381, published Feb. 20, 2014, each of which is incorporated by reference herein in its entirety.

According to various exemplary embodiments, one or more features of any one or more of the above-discussed teachings and/or exemplary embodiments may be performed or implemented using appropriately configured and/or programmed hardware and/or software elements. Determining whether an embodiment is implemented using hardware and/or software elements may be based on any number of factors, such as desired computational rate, power levels, heat tolerances, processing cycle budget, input data rates, output data rates, memory resources, data bus speeds, etc., and other design or performance constraints.

Examples of hardware elements may include processors, microprocessors, input(s) and/or output(s) (I/O) device(s) (or peripherals) that are communicatively coupled via a local interface circuit, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. The local interface may include, for example, one or more buses or other wired or wireless connections, controllers, buffers (caches), drivers, repeaters and receivers, etc., to allow appropriate communications between hardware components. A processor is a hardware device for executing software, particularly software stored in memory. The processor can be any custom made or commercially available processor, a central processing unit (CPU), an auxiliary processor among several processors associated with the computer, a semiconductor based microprocessor (e.g., in the form of a microchip or chip set), a macroprocessor, or generally any device for executing software instructions. A processor can also represent a distributed processing architecture. The I/O devices can include input devices, for example, a keyboard, a mouse, a scanner, a microphone, a touch screen, an interface for various medical devices and/or laboratory instruments, a bar code reader, a stylus, a laser reader, a radio-frequency device reader, etc. Furthermore, the I/O devices also can include output devices, for example, a printer, a bar code printer, a display, etc. Finally, the I/O devices further can include devices that communicate as both inputs and outputs, for example, a modulator/demodulator (modem; for accessing another device, system, or network), a radio frequency (RF) or other transceiver, a telephonic interface, a bridge, a router, etc.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. A software in memory may include one or more separate programs, which may include ordered listings of executable instructions for implementing logical functions. The software in memory may include a system for identifying data streams in accordance with the present teachings and any suitable custom made or commercially available operating system (0/S), which may control the execution of other computer programs such as the system, and provides scheduling, input-output control, file and data management, memory management, communication control, etc.

According to various exemplary embodiments, one or more features of any one or more of the above-discussed teachings and/or exemplary embodiments may be performed or implemented using appropriately configured and/or programmed non-transitory machine-readable medium or article that may store an instruction or a set of instructions that, if executed by a machine, may cause the machine to perform a method and/or operations in accordance with the exemplary embodiments. Such a machine may include, for example, any suitable processing platform, computing platform, computing device, processing device, computing system, processing system, computer, processor, scientific or laboratory instrument, etc., and may be implemented using any suitable combination of hardware and/or software. The machine-readable medium or article may include, for example, any suitable type of memory unit, memory device, memory article, memory medium, storage device, storage article, storage medium and/or storage unit, for example, memory, removable or non-removable media, erasable or non-erasable media, writeable or re-writeable media, digital or analog media, hard disk, floppy disk, read-only memory compact disc (CD-ROM), recordable compact disc (CD-R), rewriteable compact disc (CD-RW), optical disk, magnetic media, magneto-optical media, removable memory cards or disks, various types of Digital Versatile Disc (DVD), a tape, a cassette, etc., including any medium suitable for use in a computer. Memory can include any one or a combination of volatile memory elements (e.g., random access memory (RAM, such as DRAM, SRAM, SDRAM, etc.)) and nonvolatile memory elements (e.g., ROM, EPROM, EEROM, Hash memory, hard drive, tape, CDROM, etc.). Moreover, memory can incorporate electronic, magnetic, optical, and/or other types of storage media. Memory can have a distributed architecture where various components are situated remote from one another, but are still accessed by the processor. The instructions may include any suitable type of code, such as source code, compiled code, interpreted code, executable code, static code, dynamic code, encrypted code, etc., implemented using any suitable high-level, low-level, object-oriented, visual, compiled and/or interpreted programming language.

According to various exemplary embodiments, one or more features of any one or more of the above-discussed teachings and/or exemplary embodiments may be performed or implemented at least partly using a distributed, clustered, remote, or cloud computing resource.

According to various exemplary embodiments, one or more features of any one or more of the above-discussed teachings and/or exemplary embodiments may be performed or implemented using a source program, executable program (object code), script, or any other entity comprising a set of instructions to be performed. When a source program, the program can be translated via a compiler, assembler, interpreter, etc., which may or may not be included within the memory, so as to operate properly in connection with the O/S. The instructions may be written using (a) an object oriented programming language, which has classes of data and methods, or (b) a procedural programming language, which has routines, subroutines, and/or functions, which may include, for example, C, C++, R, Pascal, Basic, Fortran, Cobol, Perl, Java, and Ada.

According to various exemplary embodiments, one or more of the above-discussed exemplary embodiments may include transmitting, displaying, storing, printing or outputting to a user interface device, a computer readable storage medium, a local computer system or a remote computer system, information related to any information, signal, data, and/or intermediate or final results that may have been generated, accessed, or used by such exemplary embodiments. Such transmitted, displayed, stored, printed or outputted information can take the form of searchable and/or filterable lists of runs and reports, pictures, tables, charts, graphs, spreadsheets, correlations, sequences, and combinations thereof, for example.

What is claimed is:

1. A method for analyzing a tumor sample genome for genomic instability, comprising:
selectively amplifying nucleic acid sequences at targeted locations in the tumor sample genome by a targeted panel with a low sample input from a tumor sample to generate a plurality of nucleic acid sequence reads;
dividing the genome into segments having homogeneous copy numbers using log odds of heterozygous single nucleotide polymorphisms (SNPs) and copy number variation (CNV) log ratios determined for the plurality of nucleic acid sequence reads, wherein the heterozygous SNPs are distributed across the genome;
applying a threshold count to squared allelic log odds of each segment in autosomes of the genome to identify unbalanced copy number (UCN) segments;
adding numbers of bases in the UCN segments to produce a sum of UCN bases; and
dividing the sum of UCN bases by a total number of bases in all the segments identified in the autosomes of the genome to produce a ratio indicative of genomic instability.

2. The method of claim 1, wherein the ratio is expressed as a percent to give a genomic instability (GI) score.

3. The method of claim 1, further comprising applying a maximum length threshold to each of the UCN segments prior to the step of adding.

4. The method of claim 3, further comprising excluding UCN segments that span the maximum length threshold or more of a whole chromosome.

5. The method of claim 3, further comprising excluding UCN segments that span the maximum length threshold or more of a p-arm of a chromosome.

6. The method of claim 3, further comprising excluding UCN segments that span the maximum length threshold or more of a q-arm of a chromosome.

7. The method of claim 3, wherein a maximum length threshold value is a percent of a whole chromosome length, a percent of a p-arm length or a percent of a q-arm length.

8. The method of claim 7, wherein the maximum length threshold value is 90%.

9. The method of claim 1, further comprising applying a minimum length threshold to the UCN segments and excluding the UCN segments that span the minimum length threshold or less prior to the step of adding.

10. The method of claim 9, wherein the minimum length threshold is 10 megabases (Mb).

11. The method of claim 1, further comprising dividing a number of bases in the UCN segment by a total number of bases in a chromosome containing the UCN segment to produce a normalized number of UCN bases per UCN segment.

12. The method of claim 11, further comprising multiplying the normalized number of UCN bases per UCN segment by a weight value to give a weighted number of bases per UCN segment, wherein the step of adding is applied to the weighted number of bases per UCN segment for all the UCN segments to produce the sum of UCN bases.

13. The method of claim 12, wherein the weight value is a total number of UCN segments in the chromosome.

14. The method of claim 1, further comprising excluding the segments having fewer than a minimum number of heterozygous SNPs in the segment.

15. The method of claim 1, wherein the ratio indicative of genomic instability is determined based on analyzing the tumor sample only.

16. A system for analyzing a tumor sample genome for genomic instability, comprising a processor and a data store communicatively connected with the processor, the processor configured to execute instructions, which, when executed by the processor, cause the system to perform a method, including:
receiving a plurality of nucleic acid sequence reads generated by selectively amplifying nucleic acid sequences at targeted locations in the tumor sample genome by a targeted panel with a low sample input from a tumor sample;
dividing the genome into segments having homogeneous copy numbers using log odds of heterozygous single nucleotide polymorphisms (SNPs) and copy number variation (CNV) log ratios determined for the plurality of nucleic acid sequence reads, wherein the heterozygous SNPs are distributed across the genome;
applying a threshold count to squared allelic log odds of each segment in autosomes of the genome to identify unbalanced copy number (UCN) segments;
adding numbers of bases in the UCN segments to produce a sum of UCN bases; and
dividing the sum of UCN bases by a total number of bases in all the segments identified in the autosomes of the genome to produce a ratio indicative of genomic instability.

17. The system of claim 16, wherein the ratio is expressed as a percent to give a genomic instability (GI) score.

18. The system of claim 16, wherein the ratio indicative of genomic instability is determined based on analyzing the tumor sample only.

19. A non-transitory computer-readable medium storing instructions that, when executed by a computer, cause the computer to perform a method for analyzing a tumor sample genome for genomic instability, the method including:
receiving a plurality of nucleic acid sequence reads generated by selectively amplifying nucleic acid sequences at targeted locations in the tumor sample genome by a targeted panel with a low sample input from a tumor sample;
dividing the genome into segments having homogeneous copy numbers using log odds of heterozygous single nucleotide polymorphisms (SNPs) and copy number variation (CNV) log ratios determined for the plurality of nucleic acid sequence reads, wherein the heterozygous SNPs are distributed across the genome;
applying a threshold count to squared allelic log odds of each segment in autosomes of the genome to identify unbalanced copy number (UCN) segments;
adding numbers of bases in the UCN segments to produce a sum of UCN bases; and
dividing the sum of UCN bases by a total number of bases in all the segments identified in the autosomes of the genome to produce a ratio indicative of genomic instability.

20. The computer-readable medium of claim 19, wherein the ratio is expressed as a percent to give a genomic instability (GI) score.
